(19) United States
(12) Patent Application Publication
(10) Pub. No.: US 2020/0375539 A1
(43) Pub. Date: Dec. 3, 2020

(54) HEALTH PATCH UTILIZING ADHESIVE MICROSTRUCTURES

(71) Applicant: SABIC GLOBAL TECHNOLOGIES B.V., Bergen op Zoom (NL)

(72) Inventors: Manish NANDI, Malvern, PA (US); Ashir P. THAKORE, Novi, MI (US)

(21) Appl. No.: 16/604,600

(22) PCT Filed: Apr. 11, 2018
(86) PCT No.: PCT/US2018/027014

§ 371 Date: Oct. 11, 2019

Related U.S. Application Data
(60) Provisional application No. 62/484,298, filed on Apr. 11, 2017.

(51) Int. Cl. A61B 5/00; A61B 5/0408

(52) U.S. Cl. CPC A61B 5/6833; A61B 5/0205; A61B 5/04087

(57) ABSTRACT

A health patch includes a non-conductive surface configured to be disposed adjacent to skin of a subject, the non-conductive surface including a pattern of non-conductive microstructures defming protrusions and recesses, wherein the protrusions facilitate attachment of the housing to the skin of the subject.

### TECHNICAL FIELD

The disclosure concerns a health patch and more particularly, a wearable health patch utilizing adhesive microstructure technology.

### BACKGROUND

An electrocardiograph (ECG) system monitors and measures heart electrical activity in a subject over a period of time. Such measurement occurs via electrodes placed on the surface of the skin of the particular subject.

Traditionally, ECG systems utilize 12 electrode leads, with at least 10 electrodes placed at various anatomical positions on a subject to provide a complete structural and functional three-dimensional analysis of the heart. The electrode leads are used to produce electrical signals corresponding to the electrical activity generated by the heart of the subject. Such signals are generally transmitted via wiring or cable to a display which processes the signal information and converts such data into a comprehensible format for review by a health care professional.

Health care professionals have used ECG systems to monitor a subject's heart activity for years. Presently, there are a number of distinct systems that use ECG signals to monitor a subject's heart activity. These systems are not generally user-friendly, comfortable or portable, and are often cumbersome and visible to other individuals. Avoiding visibility of the system is important due to the stigma and potential embarrassment of the subject wearing the monitoring system. Thus, there exists a need to provide a cardiac monitoring system that is comfortable and portable, that may be worn under clothes without a pronounced or unnatural appearance, that produces quality and reliable data relating to the heart activity of a subject, and that uses fewer electrode patches and no wires relative to a traditional 12-lead wired ECG.

In addition, there has been interest in the fabrication and use of adhesive microstructures. Such interest derives from the gecko ability to climb smooth vertical surfaces to seemingly defy gravity. Technology developed to mirror this ability utilizes an intricate branching of fibers. These fibers are comprised of numerous micro- or nanofibers which culminate in a pad or setal area, such pad or area being in close contact with a foreign surface for attachment. Accordingly, these fiber structures confer attachment on a variety of surfaces including rough surfaces. Adhesion to surfaces by these micro- or nanofibers comes as a result of intermolecular forces such as Van der Waals' forces.

### SUMMARY

Aspects of the disclosure relate to a health patch including a non-conductive surface configured to be disposed adjacent to skin of a subject, the non-conductive surface including a pattern of non-conductive microstructures defining protrusions and recesses, wherein the protrusions facilitate attachment of the housing to the skin of the subject.

### DETAILED DESCRIPTION

The present disclosure can be understood more readily by reference to the following detailed description of the disclosure and the Examples included therein.

Before the present articles, systems, devices, and/or methods are disclosed and described, it is to be understood that they are not limited to specific synthetic methods unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

All publications mentioned herein are incorporated herein by reference to, for example, disclose and describe the methods and/or materials in connection with which the publications are cited.

### DEFINITIONS

It is also to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an electric lead patch” includes the singular patch or two or more electrode lead patches.

As used herein, the term “combination” is inclusive of different components working together, though not necessarily joined physically. Thus, for example, reference to a “combination of parts” includes, but is not limited to, the cooperation of an electrode lead patch, a wireless transmitter communication module, and a power module.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, the term “non-conductive” means not able to substantially conduct electricity (for example, less than 0.05 Siemens per meter (S/m) conductivity, less than 0.005 S/m, less than 0.002 S/m). Non-conductive may mean an insulator that does not allow a significant amount of electrical current to flow there through.

“Free of, or substantially free of” a given component indicate that the component has not been added or is not present within a given composition or system. Or, “substantially free of” may refer to less than 0.01 wt. %, or less than about 0.01 wt. %. In yet another aspect, substantially free of can be less than 100 parts per million (ppm), or less than about 100 ppm. Substantially free can refer to an amount, if present at all, below a detectable level.

As used herein, “PR interval” refers to the period, measured in milliseconds (ms), that extends from the beginning of the P wave (the onset of atrial depolarization) until the beginning of the QRS complex (the onset of ventricular depolarization) in an ECG. The PR interval may be referred to as the PQ interval.

### I. Wireless Cardiac Monitoring System

In an aspect, the present disclosure pertains to a cardiac monitoring system comprising a medical electrode lead patch, the patch comprising a housing configured to support an electrode, the housing comprising a surface configured to be disposed adjacent to skin of a subject, the first surface comprising a pattern of microstructures defining protrusions and recesses, wherein the protrusions facilitate attachment of the housing to the skin of the subject.

In another aspect, the present disclosure pertains to a medical monitoring system comprising a plurality of wireless electrode patches, each of the wireless medical electrode lead patches comprising a sensor configured to detect a medical characteristic of a subject, a wireless module configured to transmit a signal indicative of the detected medical characteristic, a power module configured to supply electrical energy to one or more of the sensor and the wireless module, and a housing configured to support an electrode, the housing comprising a surface configured to be disposed adjacent to skin of a subject, the first surface comprising a pattern of microstructures defining protrusions and recesses, wherein the protrusions facilitate attachment of the housing to the skin of the subject; and a wireless receiver configured to communicate with each of the wireless medical electrode lead patches to receive at least the signal indicative of the detected medical characteristic. In certain aspects, medical monitoring system or health monitory system comprises a plurality of wireless electrode patches which may be described as sensor patches or health patches.

In various aspects, the system of the present disclosure further comprises greater than three to five small wireless electrode patches. In other aspects, the system of the present disclosure may include locating the plurality of lead patches at different locations on the torso and limbs of the subject.

In various further aspects, the nature of the gecko type dry adhesive, or adhesive microstructures, allows for multiple attachment and detachment while maintain adhesive strength, maintaining adhesive strength in the presence of moisture, and simple, comfortable, painless removal of the adhesive from a surface, such as skin.

A. Wireless Medical Electrode Lead Patches

In one aspect, the wireless cardiac monitoring system comprises at least three wireless medical electrode lead patches. Each of the at least three wireless patches may be selectively placed at different locations on the torso or limbs of a subject. A medical electrode lead patch may comprise a thermoplastic substrate. In an aspect, an elastomeric thermoplastic material may be used. In a further aspect, the elastomeric thermoplastic material may comprise a mixture of plastic and rubber polymers, the material (i) having an ability to be stretched and an ability to return to an original shape with the removal of tension, (ii) being processable as a melt at an elevated temperature, and (iii) having an absence of significant creep, or the tendency to move slowly or deform permanently under the influence of mechanical stress.

Certain examples of thermoplastic elastomers include, but are not limited to styrenic block copolymers (TPEs), thermoplastic olefins (TPE-o), elastomeric alloys (TPV), thermoplastic polyurethanes (TPU), thermoplastic copolyester (TPE-E), and thermoplastic polyamides. In still a further aspect, the thermoplastic substrate may include a flexible silicone thermoplastic substrate may be used to encapsulate the electrode lead patch. In another aspect, a silver-silver chloride electrode may be used as a reference electrode. In this aspect, the silver-silver electrode may be used in electrochemical measurements. The silver-silver chloride electrode may comprise a silver wire that is coated by a layer of silver chloride so as to form an encapsulation of the silver wire. At one end of the electrode, a permeable body permits exposure between the surface and area to be measured and the silver chloride electrolyte. To transmit the measured data, an insulated lead wire may connect the silver wire to one or more measuring instruments. Each of the lead patches ranges from about 0.5 inches diameter to about 2 inches in diameter. Given the relatively diminutive size of the wireless medical electrode lead patches, the overall form factor and comfort of the subject will be greatly enhanced.

In an aspect, a medical electrode lead patch comprises an electrode sensor, a wireless transmitter module for communication, a power module, and a housing for adhesion.

The medical electrode lead patch functions as a conductor in collecting electrical signals from the body of the subject and transmitting those signals to a wireless receiver. More specifically, the electrode lead patch collects and transmits electrical signals from the heart of the subject.

In an aspect, each medical electrode lead patch may be labeled by name so as to avoid improper placement of the electrode lead patches on the body of the subject. In a further aspect, each medical electrode lead patch may be coded by color so as to avoid improper placement of the medical electrode lead patches on the body of the subject. In an aspect, each medical electrode patch may be labeled and coded by color so as to avoid improper placement of the electrode patches on the body of the subject.

Notably, the medical electrode lead patches must be located on the body of a subject with sufficient space between each patch so as to prevent electrical arcing across the electrode patches and potential injury to the subject and/or health care professional.

B. Electrode Sensor

As described above, each medical electrode lead patch comprises an electrode sensor along with a wireless transmitter, a power module, and a housing. Each individual wireless medical electrode lead patch contains an electrode sensor designed to detect electrical signals from each contraction, or beat of the heart, of a subject.

Electrical activity of the heart begins with spontaneous generation of an action potential by the Sinoatrial (SA) node. Such action potential transmits through the right atrium of the heart, then through Bachmann's bundle and the left atrium of the heart. This transmission activates the myocardium, or muscle cells of the atria and causes contraction of the upper chambers of the heart, and is seen as a P wave on an electrocardiograph (ECG). Such electrical activity spread through the atria travel through intermodal tracts from the SA node to the Atrioventricular (AV) node. A delay, or PR interval on an ECG, between contraction of the atria and the ventricles of the heart is rooted in the AV node, and the repolarization of the atria. The AV nodes includes the bundle of His, which splits into a right and left bundle branch, which stimulates the right and left ventricles of the heart, to contract, respectively. Specifically, each bundle branch spreads to several Purkinje fibers, which cause distinct group of ventricular muscle cells to contract. On the ECG, the contraction of the ventricles of the heart is seen in the QRS complex. Finally, the ventricles must be repolarized which are seen in the J point, ST-segment, T and U-waves on the ECG.

The potential difference between a test electrode, measuring the action potential generated by the heart, and a reference electrode, forms an electrical signal. Upon detection of such an electrical signal generated by the heart, the signal is transmitted to a wireless transmitter incorporated into the electrode lead patch

Upon detection of electrical signals generated by the heart, the signals are transmitted to a wireless transmitter incorporated into the medical electrode lead patch.

C. Wireless Transmitter

The medical electrode lead patch further comprises a wireless transmitter module in addition to an electrode, a power module, and a housing. The wireless transmitter module works cooperatively with the electrode to receive electronic signals acquired by the electrode from the heart of the subject.

In an aspect, the wireless transmitter module may comprise an application specific integrated circuit, a processor or other circuit, a plurality of signal channels, a multiplexer, an analog to digital converter (ADC), a controller, and a radio. In a further aspect, the wireless transmitter module may include different combinations or fewer of the components described above.

In an aspect, each electrode channel may comprise a filter, an amplifier, a Nyquist filter, and a track and hold circuit. The filter comprises a low pass filter for removing electromagnetic interference signals. The amplifier enhances signals from the electrodes. The Nyquist filter comprises a low pass irrelevant high frequency noise content of the amplified electric signals. Such a filter functions to enhance the reliability of the data generated and avoid measurement error. The track and hold circuit allows the system to sample from each of the channels used at the same time and avoids the potential for error when the signals from each of the channels are combined and displayed for data interpretation.

In an aspect, the multiplexer selects signals sequentially from the electrode channels using time division multiplexing. A person of ordinary skill in the art will recognize that other combination functions can be used.

In an aspect, the ADC is used to convert combined analog signals to digital signals from transmission to the receiver. In an aspect, data from the ADC may be transmitted to a device via a wireless connection. In an aspect, WiFi may be used as a wireless connection. In an alternative aspect, Bluetooth™ may be used as a wireless connection. This disclosure is not intended to limit the various wireless method to be used in transmitting data from the ADC to a device.

In an aspect, the controller may comprise a digital signal processor (DSP) that decimates the digitized signals to reduce the bandwidth necessary to transmit the electrical signal generated from the heart of the subject.

In an aspect, the radio modulates the converted digital signals with a carrier signal for transmission to the receiver.

D. Power Module

As described above, the medical electrode lead patch further comprises a power module in addition to an electrode, a wireless transmitter module, and a housing. The power module provides power to the wireless medical electrode lead patch to enable detection and transmission of electrical signals from the subject to the receiver of the cardiac monitoring system.

In an aspect, the power module is configured to supply electrical energy to the electrode sensor. In a further aspect, the power module is configured to supply electrical energy to the wireless transmitter module. In still a further aspect, the power module is configured to supply electrical energy to each of the electrode sensor and the wireless transmitter module. In effect, the power module is configured to supply electrical energy to the whole of the medical electrode lead patch.

In an aspect, the wireless cardiac monitoring system comprises a power switch to activate and deactivate the power module of any number of desired electrode patches to be used on a subject during a given time period. Thus, a power switch may activate or deactivate one, two, three, four, five, six, seven, eight, nine, ten, eleven, or even twelve medical electrode lead patches.

In an aspect, the power module is designed to house a plurality of batteries. In a further aspect, the power module utilizes a duty cycle to provide electricity and power to the system.

E. Housing

The medical electrode lead patch further comprises a housing in addition to an electrode sensor, a transmitter module, and a power module. The housing provides the medical electrode lead patch with the ability to adhere to the subject in comfort for longer periods of time during cardiac monitoring or other data generation through the device.

In an aspect, the housing of the medical electrode lead patch is configured to support an electrode, and comprises a surface configured to be disposed adjacent to skin of a subject, the first surface comprising a pattern of microstructures defining protrusions and recesses, wherein the protrusions facilitate attachment of the housing to the skin of the subject;.

In an aspect, the surface configured to be disposed adjacent to skin is formed of an elastomeric material. In a further aspect, the surface formed of an elastomer comprises a pattern of microstructures used for adhesion to a second surface.

The microstructures of the present disclosure facilitate a level of attachment to a second surface. Such microstructures comprise numerous protrusions and corresponding recesses on a given singular microstructure.

Protrusions of microstructures of the present disclosure are formed on a set of stalks that enhance the protuberance of the microstructures, and thereby facilitate adhesion at a micro or nano-level. In an aspect, the protrusions may comprise a plurality of hair-like fibers. The fibers may be non-conductive. In an aspect, the protrusions disposed on a set of stalks are arranged such that the protrusion portion of the microstructure provides adhesive strength at the surface-skin environment. In an aspect, protrusions may appear in the shape of a mushroom where the protrusion head portions are formed with a diameter in a range between about 5 μm and about 50 μm and thicknesses of between about 0.5 μm and about 4 μm. The stalk lengths of the protrusions may be formed in a range from about 15 μm to about 100 μm. Distance between protrusions may be from about 3 μm to about 5 μm. In an aspect, the bonding strength of the protrusions may be from about 15 kilopascals (kPa) to about 45 kPa. An attachment bond between the surface formed from the protrusions and the skin of the subject is at least 1 nanonewton per square meter (nN/nm²). Attachment bond strength may be measured according to a number of methods known in the art, such as for example, by determining the tensile or pull-off adhesion strength. Example standards include EN 12004, ASTM D903, or ASTM F2258.

In an aspect, protrusion structures and their corresponding stalks are formed of an elastomeric material. That is, the entire skin-facing surface of the medical electrode lead patch is formed of an elastomer.

An elastomeric thermoplastic material may comprise a mixture of plastic and rubber polymers, the material (i) having an ability to be stretched and an ability to return to an original shape with the removal of tension, (ii) being processable as a melt at an elevated temperature, and (iii) having an absence of significant creep, or the tendency to move slowly or deform permanently under the influence of mechanical stress.

In several aspects, the elastomeric material to be used may include, but is not limited to one or a mixture of styrenic block copolymers (TPEs), thermoplastic olefins (TPE-o), elastomeric alloys (TPV), thermoplastic polyurethanes (TPU), thermoplastic copolyester (TPE-E), and thermoplastic polyamides.

In some examples, the elastomeric material forming the protrusions may be formed from a material that is free of or substantially free of polydimethylsiloxane (PDMS). In yet further examples, the elastomeric material forming the protrusions may be formed from a material that is free of or substantially free of conductive carbon nanotubes or that is free of or substantially free of graphene. The elastomeric material may be formed from a material excluding graphene, carbon nanotubes, and/or polydimethylsiloxane.

Thus, due to the properties of the thermoplastic elastomeric material, the surface of the housing is pliable and form fitting to the shape of the surface to be contacted.

In a further aspect, the protrusions and corresponding microstructures provide adhesion to surfaces with different levels of smoothness. In other words, protrusions and corresponding microstructures of the present disclosure provide adhesion to both smooth and relatively rough surfaces. However, adhesion between the surface of the housing and a second surface, for instance, the skin of the subject, requires close proximity.

In an aspect, inclusion of additional protrusions and corresponding stalks at different lengths provides an increased strength of adhesion. Thus, the addition of a set of protrusions and corresponding stalks formed of an elastomeric materials to an previously formed set of protrusions and corresponding stalks will result in a an increased strength of adhesions based on the increased surface contact between the protrusions and the surface of the subject.

In yet another aspect, the housing may include a ventral surface, configured to include microstructures and corresponding protrusions and stalks located adjacent to the surface, or skin of the subject. The ventral surface is further configured to facilitate attachment to the surface, or skin of the subject. Furthermore, the housing may include a dorsal surface, configured to include microstructures and corresponding protrusions and stalks located adjacent, or directly on top of to the ventral surface of the housing. The dorsal surface is further configured to facilitate attachment to a secondary dorsal surface.

In an aspect, the adhesive properties of the protrusions and corresponding microstructures allow for multiple attachment and detachment to different surfaces while providing sufficient adhesion to secure the medical electrode lead patch to the subject. In yet a further aspect, the protrusions and corresponding microstructures maintain adhesive properties despite introduction of fluids such as water.

In certain aspects, the housing includes materials, such as but not limited to the thermoplastic elastomeric materials described herein, such that the housing is non-conductive.

### II. Wireless Receiver

As described above, the wireless cardiac monitoring system further comprises a wireless receiver in addition to a plurality of wireless medical electrode lead patches.

In an aspect the wireless receiver comprises a radio, a controller, a digital to analog converter (DAC), a demultiplexer, a transceiver, and a plurality of electrode signal channels.

The radio functions to demodulate received signals for identifying data generated from the combined electrode signals originating from the various medical electrode lead patches located at different locations on the subject.

The controller functions to control operation of the various components of the receiver including the ability to control or further process signals from the radio. In an aspect, the controller may convert received signals to digital information or interpolate data transmitted from the medical electrode lead patches. Such functions are exemplary, but are in no way meant to be an exhaustive list of operations a controller may perform.

In an aspect, the controller interpolates signals from the electrode lead patches to return the effective sample rate from about 25 hertz (Hz) to about 1 kHz or another frequency.

The DAC functions to convert digital signals to analog signals.

The demultiplexer functions to separate the individually regenerated signals onto a separate electrode signal channel for each regenerated signal. Thus, a regenerated signal will be separated onto an electrode signal channel for each of the medical electrode lead patches generating data from the heart of the subject.

The transceiver functions to both transmit and receive signals in accordance with communicated with the wireless transmitter module.

In an aspect, the wireless receiver has as many electrode signal channels as there are wireless medical electrode lead patches. That is, for every electrode lead patch used on a subject, the wireless receiver has a corresponding electrode signal channel.

The electrode signal channel comprises a sample and hold circuit, a filter, and an attenuator.

The sample and hold circuit is operated by the controller such that the converted electrode signals from each of the wireless medical electrode lead patches appear concurrently on each of the electrode signal channels.

The filter may comprise a low pass reconstruction filter operating to remove high frequency noise associated with the DAC or other conversion process.

The attenuator comprises an amplifier used to reduce the amplitude of the electrode signals to a level associated with electrode signals previously amplified by the transmitter module.

In an aspect, the receiver may be attached to the subject undergoing the cardiac monitoring. Attachment to the subject may include the use of wiring, cables, etc.

In a further aspect, the receiver may be close to the body of the subject, but not attached.

### Display Module

Upon receipt of the electrical signals from the system, the signals are converted to readable data and presented on a medium. In an aspect, the readable data to be presented on a medium is a rendering of a heart and the cardiac activity of a subject. Such a rendering displays the entire image of the heart so as to give a full view of the cardiac activity of the subject.

In a further aspect, the data presented on a medium is to be interpreted by health care professionals or the subject undergoing measurement. In alternative aspects, such data may be analyzed and interpreted by various healthcare or medical workers with an interest in the cardiac activity of the measured subject.

In several aspects, signals are transmitted to wireless devices and converted into data to be analyzed and interpreted. In an aspect, such data may be transmitted wirelessly for analysis and interpretation to a smart phone. In a further aspect, data from the system may be transmitted and presented on a personal computer (PC). In yet a further aspect, such data from the system by may be transmitted and presented on a tablet, or any other type of personal electronic device used for data storage and/or presentation.

Placement of the Wireless Medical Electrode Lead Patches

As described above, the present disclosure relates to a wireless cardiac monitoring system including multiple wireless medical electrode lead patches. In an aspect, the system comprises three medical electrode patches. In a further aspect, the system may comprise four medical electrode lead patches. In still a further aspect, the system comprises five medical electrode lead patches. In yet a further aspect, the system may comprise six medical electrode lead patches. In still further aspects, the system may comprise seven, eight, nine, ten, eleven, or twelve medical electrode lead patches.

Traditional cardiac monitoring via electrocardiography utilizes at least 10 electrodes placed at different locations to obtain the most accurate information about the structure and function of the heart of the subject. However, using the systems of the present disclosure, wireless electrode patches may be selectively placed on a patient to determine a complete ECG using a customized (for example, minimized) number of electrodes. A complete ECG may be defined as an ECG readout or trace representing a normal sinus rhythm and may comprise at least a discernable P wave, QRS complex, and T wave. Additionally, the complete ECG may comprise PR interval, J-point, ST segment, and U wave. It is understood that other portions of the ECG may be includes such as a corrected QT interval. It is further understood that noise or artifacts may be represented in the ECG trace and may be distinguished from the complete trace, as defined above. Additionally or alternatively, a complete ECG may be represented by one or more predetermined characteristic traces such as arrhythmias, including for example, characteristic traces representing atrial fibrillation, atrial flutter, ventricular flutter, and/or ventricular tachycardia. Other characteristic traces may be known and may be catalogued for comparison to determine a discernable complete ECG representative of a match to a characteristic trace.

In order to determine the select number of electrodes and placement of the select number of electrodes in a customized manner, various learning mechanism may be used. For example, heuristics, machine learning, historical patient data, and other learning mechanisms may be used to determine a select number and placement of the wireless electrode patches of the present disclosure. The select number of wireless electrode patches may be optimized to be the minimum number of wireless electrode patches required to produce a complete ECG trace. In certain aspects, the select number of wireless electrode patches may be less than the conventional 12 leads or 10 placed electrodes. As such, the form factor of the wireless electrode patches and the minimized number of the wireless electrode patches provide a complete ECG will minimize intrusiveness to the patient.

Medical electrodes may be placed at a location on the right arm of the subject (RA), the same location on the left arm of the subject (LA), the right calf (RL), the same location of the left calf (LL), in the fourth intercostal space between rib 4 and rib 5 and immediately to the right of the sternum of the subject (V₁), in the fourth intercostal space between rib 4 and rib 5 and immediately to the left of the sternum of the subject (V₂), between V₁ and V₂ (V₃), in the fifth intercostal space between ribs 5 and 6 in the mid-clavicular line (V₄), horizontally even with V₄ in the left anterior axillary line (V₅), and horizontally even with V₄ and V₅ in the midaxillary line (V₆).

The wireless cardiac monitoring system of the present disclosure utilizes greater than about three to about five wireless medical electrode lead patches to monitor the structural and functional characteristics of the heart of a subject. In an aspect, each of these wireless electrode patches may be placed at a location correlating to any one of RA, LA, RL, LL, and V₁-V₆.

In an aspect, six wireless electrode patches may be placed at various locations on the subject. In a further aspect, the six wireless electrode patches may be placed at (i) a location in the fourth intercostal space between rib 4 and rib 5 and immediately to the right of the sternum of the subject (V₁), (ii) a location in the fourth intercostal space between rib 4 and rib 5 and immediately to the left of the sternum of the subject (V₂), (iii) a location between V₁ and V₂ (V₃), (iv) a location in the fifth intercostal space between ribs 5 and 6 in the mid-clavicular line (V₄), (v) a location horizontally even with V₄ in the left anterior axillary line (V₅), and (vi) a location horizontally even with V₄ and V₅ in the midaxillary line (V₆).

In an aspect, ten wireless electrode patches may be placed at various locations on the subject. In a further aspect, the ten wireless electrode patches may be placed at (i) a location on the right arm of the subject (RA), (ii) the same location on the left arm of the subject (LA), (iii) a location on the right calf (RL), (iv) the same location of the left leg/calf (LL), (v) a location in the fourth intercostal space between rib 4 and rib 5 and immediately to the right of the sternum of the subject (V₁), (vi) a location in the fourth intercostal space between rib 4 and rib 5 and immediately to the left of the sternum of the subject (V₂), (vii) a location between V₁ and V₂ (V₃), (viii) a location in the fifth intercostal space between ribs 5 and 6 in the mid-clavicular line (V₄), (ix) a location horizontally even with V₄ in the left anterior axillary line (V₅), and (x) a location horizontally even with V₄ and V₅ in the midaxillary line (V₆).

In an aspect, between three and five wireless electrode patches may be placed at various locations on the subject. In a further aspect, the plurality of wireless electrode patches may be placed at any of three to five locations including, but not limited to (i) a location in the fourth intercostal space between rib 4 and rib 5 and immediately to the right of the sternum of the subject (Vi), (ii) a location in the fourth intercostal space between rib 4 and rib 5 and immediately to the left of the sternum of the subject (V2), (iii) a location between V₁ and V₂ (V₃), (iv) a location in the fifth intercostal space between ribs 5 and 6 in the mid-clavicular line (V₄), and (v) a location horizontally even with V₄ in the left anterior axillary line (V₅).

In an aspect, the wireless cardiac monitoring system may be used to measure other vital medical characteristics including body temperature. In a further aspect, the wireless cardiac monitoring system may be used to measure pulse rate. In still a further aspect, the wireless cardiac monitoring system may be used to measure heart rate. In yet a further aspect, the wireless cardiac monitoring system may be used to measure respiration rate. In another aspect, the wireless cardiac monitoring system may be used to measure electroencephalography EEG signals. In still another aspect, the wireless cardiac monitoring system may be used to measure pulse oximeter signals.

In addition to improving comfort and overall appearance of the subject undergoing cardiac monitoring, the overall quality of data is comparable between the present disclosure comprising fewer electrodes and a traditional 12-lead electrocardiogram.

### ASPECTS

Aspect 1A. A sensor patch comprising a housing configured to support a sensor, the housing comprising a surface configured to be disposed adjacent to skin of a subject, the surface comprising a pattern of microstructures defining protrusions and recesses, wherein the protrusions facilitate attachment of the housing to the skin of the subject.

Aspect 1B. A sensor patch consisting essentially of a housing configured to support a sensor, the housing comprising a surface configured to be disposed adjacent to skin of a subject, the surface comprising a pattern of microstructures defining protrusions and recesses, wherein the protrusions facilitate attachment of the housing to the skin of the subject.

Aspect 1C. A sensor patch consisting a housing configured to support a sensor, the housing comprising a surface configured to be disposed adjacent to skin of a subject, the surface comprising a pattern of microstructures defining protrusions and recesses, wherein the protrusions facilitate attachment of the housing to the skin of the subject.

Aspect 2. The sensor patch of aspects 1A-C, wherein the protrusions comprise a plurality of non-conductive polymeric hair-like fibers.

Aspect 3. The sensor patch of any one of aspects 1A-2, wherein the protrusions are formed from a material excluding conductive carbon nanotubes and graphene.

Aspect 4. The sensor patch of any one of aspects 1A-2, wherein the protrusions are formed from a material excluding conductive carbon nanotubes.

Aspect 5. The sensor patch of any one of aspects 1A-2, wherein the protrusions are formed from a material excluding graphene.

Aspect 6. The sensor patch of any one of aspects 1A-5, wherein the protrusions are formed from a material comprising thermoplastic elastomer, thermoplastic polyurethane, silicone, hybrid thermoplastic polyurethane (TPU) and a fully crosslinked silicone rubber, liquid silicone rubber.

Aspect 7. The sensor patch of any one of aspects 1A-6, wherein the protrusions are formed from a material comprising polydimethylsiloxane (PDMS).

Aspect 8. The sensor patch of any one of aspects 1A-7, wherein the protrusions facilitate attachment without a chemical adhesive.

Aspect 9. The sensor patch of any one of aspects 1A-8, wherein the surface defines an aperture, and wherein at least a portion of the sensor is disposed in the aperture.

Aspect 10. The sensor patch of aspect 9, wherein the portion of the sensor disposed in the aperture is at least partially conductive and is configured to contact the skin of the subject.

Aspect 11. The sensor patch of any one of aspects 1A-10, wherein the sensor is configured to measure a pulse rate of the subject.

Aspect 12. The sensor patch of any one of aspects 1A-11, wherein the sensor is configured to measure a heart rate of the subject.

Aspect 13. The sensor patch of any one of aspects 1A-12, wherein the sensor is configured to measure a respiration rate of the subject.

Aspect 14. The sensor patch of any one of aspects 1A-13, wherein the sensor is configured to measure a body temperature of the subject.

Aspect 15. The sensor patch of any one of aspects 1A-14, wherein the sensor is configured to measure an EEG signaling of the subject.

Aspect 16. The sensor patch of any one of aspects 1A-15, wherein the sensor is configured to measure a pulse oximeter signaling of the subject.

Aspect 17. The sensor patch of any one of aspects 1A-16, wherein the housing is non-conductive.

Aspect 18A. A health patch comprising a non-conductive surface configured to be disposed adjacent to skin of a subject, the non-conductive surface comprising a pattern of non-conductive microstructures defining protrusions and recesses, wherein the protrusions facilitate attachment of the housing to the skin of the subject.

Aspect 18B. A health patch consisting essentially of a non-conductive surface configured to be disposed adjacent to skin of a subject, the non-conductive surface comprising a pattern of non-conductive microstructures defining protrusions and recesses, wherein the protrusions facilitate attachment of the housing to the skin of the subject.

Aspect 18C. A health patch consisting of a non-conductive surface configured to be disposed adjacent to skin of a subject, the non-conductive surface comprising a pattern of non-conductive microstructures defining protrusions and recesses, wherein the protrusions facilitate attachment of the housing to the skin of the subject.

Aspect 19. The health patch of aspects 18A-18C, wherein the protrusions comprise a plurality of polymeric hair-like fibers.

Aspect 20. The health patch of any one of aspects 18A-19, wherein the protrusions are formed from a material excluding conductive carbon nanotubes and graphene.

Aspect 21. The health patch of any one of aspects 18A-19, wherein the protrusions are formed from a material excluding conductive carbon nanotubes.

Aspect 22. The health patch of any one of aspects 18A-19, wherein the protrusions are formed from a material excluding graphene.

Aspect 23. The health patch of any one of aspects 18A-22, wherein the protrusions are formed from a material comprising thermoplastic elastomer, thermoplastic polyurethane, silicone, hybrid thermoplastic polyurethane (TPU) and a fully crosslinked silicone rubber, liquid silicone rubber.

Aspect 24. The health patch of any one of aspects 18A-23, wherein the protrusions are formed from a material excluding polydimethylsiloxane (PDMS).

Aspect 25. The health patch of any one of aspects 18A-24, wherein the protrusions facilitate attachment without a chemical adhesive.

Aspect 26. The health patch of any one of aspects 18A-25, further comprising a microneedle disposed adjacent the non-conductive surface.

Aspect 27. The health patch of any one of aspects 18A-26, further comprising an absorbent bandage material adjacent the non-conductive surface.

Aspect 28. The health patch of any one of aspects 18A-27, wherein an attachment bond between the non-conductive surface and the skin of the subject is at least 1 nN/nm².

Aspect 29A. A health monitoring system comprising:

a plurality of wireless electrode patches, each of the wireless electrode patches comprising a sensor configured to detect a medical characteristic of a subject, a wireless module configured to transmit a signal indicative of the detected medical characteristic, a power module configured to supply electrical energy to one or more of the sensor and the wireless module, and a housing configured to support an electrode, the housing comprising a surface configured to be disposed adjacent to skin of a subject, the surface comprising a pattern of microstructures defining protrusions and recesses, wherein the protrusions facilitate attachment of the housing to the skin of the subject; and a wireless receiver configured to communicate with each of the wireless electrode patches to receive at least the signal indicative of the detected medical characteristic.

Aspect 29B. A health monitoring system consisting essentially of:

a plurality of wireless electrode patches, each of the wireless electrode patches comprising a sensor configured to detect a medical characteristic of a subject, a wireless module configured to transmit a signal indicative of the detected medical characteristic, a power module configured to supply electrical energy to one or more of the sensor and the wireless module, and a housing configured to support an electrode, the housing comprising a surface configured to be disposed adjacent to skin of a subject, the surface comprising a pattern of microstructures defining protrusions and recesses, wherein the protrusions facilitate attachment of the housing to the skin of the subject; and a wireless receiver configured to communicate with each of the wireless electrode patches to receive at least the signal indicative of the detected medical characteristic.

Aspect 29C. A health monitoring system consisting of:

a plurality of wireless electrode patches, each of the wireless electrode patches comprising a sensor configured to detect a medical characteristic of a subject, a wireless module configured to transmit a signal indicative of the detected medical characteristic, a power module configured to supply electrical energy to one or more of the sensor and the wireless module, and a housing configured to support an electrode, the housing comprising a surface configured to be disposed adjacent to skin of a subject, the surface comprising a pattern of microstructures defining protrusions and recesses, wherein the protrusions facilitate attachment of the housing to the skin of the subject; and

a wireless receiver configured to communicate with each of the wireless electrode patches to receive at least the signal indicative of the detected medical characteristic.

Aspect 30. The health monitoring system of aspects 29A-29C, wherein the protrusions comprise a plurality of polymeric hair-like fibers.

Aspect 31. The health monitoring system of any one of aspects 29A-30, wherein the protrusions are formed from a material excluding conductive carbon nanotubes and graphene.

Aspect 32. The health monitoring system of any one of aspects 29A-30, wherein the protrusions are formed from a material excluding conductive carbon nanotubes.

Aspect 33. The health monitoring system of any one of aspects 29A-30, wherein the protrusions are formed from a material excluding graphene.

Aspect 34. The health monitoring system of any one of aspects 29A-30, wherein the protrusions are formed from a material comprising silicone.

Aspect 35. The health monitoring system of any one of aspects 29A-30, wherein the protrusions are formed from a material comprising polydimethylsiloxane (PDMS).

Aspect 36. The health monitoring system of any one of aspects 29A-35, wherein the protrusions facilitate attachment without a chemical adhesive.

Aspect 37. The health monitoring system of any one of aspects 29A-36, wherein the surface of the housing defines an aperture, and wherein at least a portion of the sensor is disposed in the aperture.

Aspect 38. The health monitoring system of aspect 37, wherein the portion of the sensor disposed in the aperture is at least partially conductive and is configured to contact the skin of the subject.

Aspect 39. The health monitoring system of any one of aspects 29A-38, wherein the surface of the housing is non-conductive.

Aspect 40. The health monitoring system of any one of aspects 29A-39, wherein the detected medical characteristic comprises one or more electrical signals indicative of an activity of a heart of a user of the medical monitoring system.

Aspect 41. The health monitoring system of any one of aspects 29A-40, wherein the output comprises a complete electrocardiogram trace.

Aspect 42. The health monitoring system of any one of aspects 29A-41, wherein a placement of the plurality of wireless electrode patches is customized for a user of the medical monitoring system.

Aspect 43. The health monitoring system of any one of aspects 29A-42, wherein the plurality of wireless electrode patches comprises six wireless electrode patches.

Aspect 44. The health monitoring system of aspect 43, wherein the location of the six wireless electrode patches includes a location in the fourth intercostal space between rib 4 and rib 5 and immediately to the right of the sternum of the subject (V₁), a location in the fourth intercostal space between rib 4 and rib 5 and immediately to the left of the sternum of the subject (V₂), a location between V₁ and V₂ (V₃), a location in the fifth intercostal space between ribs 5 and 6 in the mid-clavicular line (V₄), and a location horizontally even with V₄ in the left anterior axillary line (V₅), and a location horizontally even with V₄ and V₅ in the midaxillary line (V₆).

Aspect 45. The health monitoring system of any one of aspects 29A-42, wherein the plurality of wireless electrode patches comprises less than ten wireless electrode patches.

### EXAMPLES

In an aspect, the wireless cardiac monitoring system is used to measure structural and functional medical characteristics of the heart of a subject. First, a plurality of wireless medical electrode lead patches is placed at various anatomical locations on the skin of a subject. Such locations may include, but are not limited to no fewer than three of a location on the right arm of the subject (RA), the same location on the left arm of the subject (LA), the right calf (RL), the same location of the left calf (LL), in the fourth intercostal space between rib 4 and rib 5 and immediately to the right of the sternum of the subject (V₁), in the fourth intercostal space between rib 4 and rib 5 and immediately to the left of the sternum of the subject (V₂), between V₁ and V₂ (V₃), in the fifth intercostal space between ribs 5 and 6 in the mid-clavicular line (V₄), horizontally even with V₄ in the left anterior axillary line (V₅), and horizontally even with V₄ and V₅ in the midaxillary line (V₆).

Upon securing the plurality of wireless medical electrode lead patches to the skin of the subject, measurement of medical characteristics may begin. Each individual wireless medical electrode lead patch contains an electrode sensor designed to detect electrical signals from each contraction, or beat of the heart of a subject. The individual wireless medical electrode lead patch comprises a wireless transmitter module and a power module in addition to the electrode sensor. Cardiac monitoring and measurement of characteristics may begin upon activation of the wireless medical electrode lead patch through the power module supplying electrical energy to the entirety of the patch. Electrical signals generated by the heart of the subject are detected by the electrode sensor of the monitoring system and transfers these signals to the wireless transmitter module portion of the wireless medical electrode patch. Upon detection of electrical signals from the heart, the transmitter module processes the signaling in a variety of ways before relaying the electrical signal to the wireless receiver via radio transmission. Such radio transmission occurs between the wireless transmitter module and the wireless receiver of the cardiac monitoring system. Upon receiving the signaling from the transmitter, the wireless receiver processes, filters, and converts the electrical signals from the heart of the patient from raw data into a comprehensible format for review by a healthcare professional or by the subject himself

In some aspects the systems, patches, sensors, and associated components described herein are suitable for use in any applicable medical and/or healthcare-related application. Exemplary applications include, but are not limited to, general healthcare delivery, diagnostic applications, therapeutic applications, and drug delivery applications.

It is to be understood that any feature described in relation to any one aspect or Example may be used alone, or in combination with other features described, and may also be used in combination with one or more features of any other of the aspects or Examples, or any combination of any other of the aspects and examples. Further, equivalents and modifications not described above may also be employed without departing from the scope of the disclosure, which is defined in the accompanying claims.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein can be different from the actual publication dates, which can require independent confirmation.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

While typical aspects have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A health patch comprising a non-conductive surface configured to be disposed adjacent to skin of a subject, the non-conductive surface comprising a pattern of non-conductive microstructures defining protrusions and recesses, wherein the protrusions facilitate attachment of the health patch to the skin of the subject.

2. The health patch of claim 1, wherein the protrusions comprise a plurality of polymeric hair-like fibers.

3. The health patch of claim 1, wherein the protrusions are formed from a material excluding conductive carbon nanotubes and graphene.

4. The health patch of claim 1, wherein the protrusions are formed from a material excluding conductive carbon nanotubes.

5. The health patch of claim 1, wherein the protrusions are formed from a material excluding graphene.

6. The health patch of claim 1, wherein the protrusions are formed from a material comprising thermoplastic elastomer, thermoplastic polyurethane, silicone, hybrid thermoplastic polyurethane (TPU) and a fully crosslinked silicone rubber, liquid silicone rubber.

7. The health patch of claim 1, wherein the protrusions are formed from a material excluding polydimethylsiloxane (PDMS).

8. The health patch of claim 1, wherein the protrusions facilitate attachment without a chemical adhesive.

9. The health patch of claim 1, further comprising a microneedle disposed adjacent the non-conductive surface.

10. The health patch of claim 1, further comprising an absorbent bandage material adjacent the non-conductive surface.

11. The health patch of claim 1, wherein an attachment bond between the non-conductive surface and the skin of the subject is at least 1 nN/nm².

12. A health monitoring system consisting of:
a plurality of wireless electrode patches, each of the wireless electrode patches comprising a sensor configured to detect a medical characteristic of a subject, a wireless module configured to transmit a signal indicative of the detected medical characteristic, a power module configured to supply electrical energy to one or more of the sensor and the wireless module, and a housing configured to support an electrode, the housing comprising a surface configured to be disposed adjacent to skin of a subject, the surface comprising a pattern of microstructures defining protrusions and recesses, wherein the protrusions facilitate attachment of the housing to the skin of the subject; and
a wireless receiver configured to communicate with each of the wireless electrode patches to receive at least the signal indicative of the detected medical characteristic.

13. The health monitoring system of claim 12, wherein the protrusions comprise a plurality of polymeric hair-like fibers.

14. The health monitoring system of claim 12, wherein the protrusions are formed from a material excluding conductive carbon nanotubes and graphene.

15. The health monitoring system of claim 12, wherein the protrusions are formed from a material excluding conductive carbon nanotubes.

16. The health monitoring system of claim 12, wherein the protrusions are formed from a material excluding graphene.

17. The health monitoring system of claim 12, wherein the protrusions are formed from a material comprising silicone.

18. The health monitoring system of claim 12, wherein the protrusions are formed from a material comprising polydimethylsiloxane (PDMS).

19. The health monitoring system of claim 12, wherein the protrusions facilitate attachment without a chemical adhesive.

20. The health monitoring system of claim 12, wherein the surface of the housing defines an aperture, and wherein at least a portion of the sensor is disposed in the aperture.
